(19) United States
(12) Patent Application Publication
(10) Pub. No.: US 2025/0032552 A1
(43) Pub. Date: Jan. 30, 2025

(54) METHOD OF TREATING SEVERE GRAFT VERSUS HOST DISEASE

(71) Applicant: MESOBLAST INTERNATIONAL SÁRL, Meyrin (CH)

(72) Inventor: Silviu ITESCU, Melbourne (AU)

(21) Appl. No.: 18/723,204

(22) PCT Filed: Dec. 23, 2022
(86) PCT No.: PCT/IB2022/062711

§ 371 Date: Jun. 21, 2024

(30) Foreign Application Priority Data
Dec. 23, 2021 (AU) 2021904222

(51) Int. Cl. A61K 35/28; G01N 33/68

(52) U.S. Cl. CPC A61K 35/28; G01N 33/6893; G01N 2800/245

(57) ABSTRACT

The present disclosure relates to mesenchymal lineage precursor or stem cell mediated methods for treating severe Graft versus Host Disease (GvHD) in subjects at high risk of poor outcomes.

### TECHNICAL FIELD

The present disclosure relates to mesenchymal lineage precursor or stem cell mediated methods for treating severe Graft versus Host Disease (GvHD) in subjects at high risk of poor outcomes.

### BACKGROUND

Acute and chronic Graft versus Host Disease (GvHD) are immunological disorders that are the major cause of nonrelapse mortality (NRM) after allogeneic stem cell transplantation. Acute GvHD affects 40% to 60% of patients and targets the skin, liver, and gastrointestinal tract.

Recently, the Mount Sinai Acute GvHD International Consortium (MAGIC) Algorithm Probability has been reported. The (MAGIC) Algorithm Probability combines the serum concentration of two biomarkers into a single value that predicts long-term outcomes such as response to therapy after 28 days and 6-month NRM. GvHD patients with high MAP are considered to have the most severe disease and poor outcomes. In particular, these patients have the lowest probability of achieving clinical response to therapy, and a low overall 6-month survival rate of 20%.

Clearly, there is an unmet therapeutic need in the art for treating severe GvHD, in particular in subjects at high risk of poor outcomes.

### SUMMARY

The present inventors have surprisingly identified that subjects with severe GvHD can be treated with mesenchymal lineage precursor or stem cells (MLPSCs). Accordingly, in a first example, the present disclosure relates to a method for treating Graft versus Host Disease (GvHD) in a subject, the method comprising administering to the subject a composition comprising MLPSCs, wherein the subject has severe GvHD as determined by MAGIC Algorithm Probability (MAP). In another example, the present disclosure relates to a method of selecting Graft versus Host Disease (GvHD) patients for treatment with MLPSCs, the method comprising i) determining or having determined a subject's MAGIC Algorithm Probability (MAP) ii) selecting a subject having MAP indicative of severe GvHD for treatment, preferably, wherein the treatment comprises administering a composition comprising MLPSCs. In another example, the present disclosure relates to a method of reducing the risk of mortality in a subject with Graft versus Host Disease (GvHD), wherein the subject has severe GvHD as measured by MAGIC Algorithm Probability (MAP), the method comprising administering to the subject a composition comprising MLPSCs, preferably wherein mortality is 6-month non-relapse mortality (NRM). In an example, the subject's MAP is greater than (>) 0.2. In an example, the subject's MAP is greater than or equal to (≥) 0.29. In another example, the subject's MAP is between 0.2 and 0.35. In another example, the subject's MAP is ≥0.291.

In another example, the subject is steroid refractory. In another example, subject has acute Graft versus Host Disease (aGvHD). In another example, the subject is a paediatric subject.

In another example, the subject has very low probability of achieving a clinical response to primary therapy within 28 days and/or has a high risk of 6 month non-relapse mortality. In another example, the subject has a 6 month non-relapse mortality risk of ≥70%, ≥80%, or ≥90%. In another example, the subject has worsened within 3 days of primary therapy or not responded within 7 days of a primary therapy. For example, the primary is systemic steroids.

In another example, the subject has severe GvHD as classified by one or more of the following:
Grade B, C, or D according to the IBMTR severity scale;
Grade II GvHD according to the Glucksberg severity scale;
Grade IIII/V GvHD according to the Glucksberg severity scale;
Minnesota high risk GvHD.

In another example, the subject has multi-organ involvement.

In another example, the subject has at least a partial response after 28 days of treatment. In another example, the subject has at least a partial response at least 28 to 180 days after treatment. For example, partial response is characterized by one or more or all of:
Reduction in Skin % BSA score of at least one point;
Reduction in mouth score of at least one point;
Reduction in eye score of at least one point;
Reduction in skin features score of at least one point;
Reduction in gastrointestinal tract score of at least one point;
Reduction in liver score of at least one point;
Reduction in lung symptom score of at least one point;
Reduction in lung FEV1 score of at least one point;
Reduction in joints and fascia score of at least one point;
Reduction in genital tract score of at least one point.

In another example, a partial response is characterized by one or more or all of:
Reduction in Skin % BSA score of at least one point;
Reduction in mouth score of at least one point;
Reduction in eye score of at least one point.

In an example, treatment with compositions of the present disclosure increases the probability of the subject's survival. In an example, treatment increases the probability of the subject surviving for at least 100 days after initiation of treatment. In an example, treatment increases the probability of the subject surviving for at least 180 days. In an example, treatment decreases the subject's risk of 6 month non-relapse mortality to between 40% and 80%. In an example, treatment decreases the subject's risk of 6 month non-relapse mortality to at least 60%.

In an example, the subject's risk of 6 month non-relapse mortality is decreased relative to a subject who does not receive MLPSCs. In an example, the subject that does not receive MLPSCs receives best available therapy. In an example, the best available therapy is selected from one of more of the group consisting of extracorporeal photopheresis, etanercept, infliximab, ruxolitinib, anti-thymocyte globulin, mycophenolate, alemtuzumab, basiliximab, and tocilizumab.

The present inventors have also surprisingly identified that treatment with MLPSCs results in durable reduction in ST2 and MAP. Accordingly, in an example, treatment with compositions of the present disclosure reduces the subject's ST2 levels. In another example, treatment with compositions of the present disclosure reduces the subject's MAP level. In an example, treatment reduces the subject's MAP by day 28. In an example, treatment reduces the subject's MAP to less than (<) 0.29. In another example, treatment reduces the subject's MAP to <0.29 by day 100. In an example, the reduction in the subject's MAP is sustained at day 100. In another example, the reduction in the subject's MAP is sustained at day 160. In another example, the reduction in the subject's MAP is sustained at day 180. In another example, treatment reduces the subject's MAP to <0.291.

In an example, treatment also reduces the level of an inflammatory biomarker selected from the group consisting of ELAFIN, sIL-2ra, TNFR1, IL-8, HGF.

In an example, methods of the present disclosure comprises the steps of:
i) determining or having determined the subject's Reg3α and ST2 levels;
ii) calculating or having calculated the subject's MAP according to the subject's Reg3α and ST2 levels;
iii) selecting a subject for treatment with MLPSCs, wherein the subject's MAP is ≥0.29; and
iv) administering to the subject a composition comprising MLPSCs.

In an example, the MLPSCs are STRO-1+. In an example, MLPSCs are allogeneic. In an example, the cells are culture expanded. In an example, the cells are TNAP+ before they are culture expanded. In an example, the cells have been cryopreserved. In an example, the cells are administered in amount between 1×10⁷ and 2×10⁸ cells. In an example, the composition further comprises Plasma-Lyte A, dimethyl sulfoxide (DMSO), human serum albumin (HSA). In an example, the composition further comprises Plasma-Lyte A (70%), DMSO (10%), HSA (25%) solution, the HSA solution comprising 5% HSA and 15% buffer. In an example, the composition comprises greater than 6.68×10⁶ viable cells/mL. In an example, the composition comprises human bone marrow-derived allogeneic mesenchymal precursor cells (MPCs). In an example, MPCs are isolated from bone mononuclear cells with anti-STRO-3 antibodies, expanded ex vivo, and cryopreserved. In an example, the MLPSCs are mesenchymal stem cells (MSCs).

In an example, the subject has previously received another therapy. In an example, the therapy is selected from the group consisting of steroids, extracorporeal photopheresis, etanercept, infliximab, ruxolitinib, anti-thymocyte globulin, mycophenolate, alemtuzumab, basiliximab, and tocilizumab.

In an example, the composition is administered to the gastrointestinal tract wall of the subject. In an example, the composition is administered to the submucosal layer of the subject's gastrointestinal tract wall. In an example, the composition is administered to a site of inflammation in the subject's gastrointestinal tract wall. In an example, the composition is administered intravenously. In an example, the subject receives at least two doses. In another example, the subject receives at least 2, 3, 4, 5, 6, 7, 8, 9 or 10 doses. In an example the first two doses are administered weekly for two weeks. In another example the first two doses are administered weekly every two weeks. In an example, the third and subsequent doses are administered monthly.

### BRIEF DESCRIPTION OF DRAWINGS

FIG. 1: Survival at day +180 for patients with a high-risk MAP treated with cell therapy compared to high-risk control patients who received best available therapy.

FIG. 2: Clinical response and survival at day 28 for patients with a high-risk MAP treated with cell therapy compared to high-risk control patients who received best available therapy.

FIG. 3: Cell therapy treated children with baseline MAP ≥0.29 is associated with decreased MAP levels over 28 days.

FIG. 4: Biomarker Evidence of in vivo activity. Treatment with cell therapy results in durable overall reduction in soluble ST2 Levels and MAGIC Biomarker Score (MBS, MAP).

FIG. 5: Level of IL2R inhibition by stem cells correlates with reduction over 28 days in circulating ST2 levels and MAP scores in acute GvHD patients.

FIG. 6: Longitudinal analysis of biomarkers by baseline MAP: Cell therapy results in durable reduction of high inflammatory biomarkers in patients with MAP ≥0.29 to levels in those with MAP <0.29.

### DETAILED DESCRIPTION

### General Techniques and Definitions

Unless specifically defined otherwise, all technical and scientific terms used herein shall be taken to have the same meaning as commonly understood by one of ordinary skill in the art (e.g., in cell culture, molecular biology, stem cell culture, immunology, clinical studies, general medicine, and biochemistry).

Unless otherwise indicated, cell culture techniques and assays utilized in the present disclosure are standard procedures, well known to those skilled in the art. Such techniques are described and explained throughout the literature in sources such as, J. Perbal, A Practical Guide to Molecular Cloning, John Wiley and Sons (1984), J. Sambrook et al., Molecular Cloning: A Laboratory Manual, Cold Spring Harbour Laboratory Press (1989), T. A. Brown (editor), Essential Molecular Biology: A Practical Approach, Volumes 1 and 2, IRL Press (1991), D. M. Glover and B. D. Hames (editors), and F. M. Ausubel et al. (editors), Current Protocols in Molecular Biology, Greene Pub. Associates and Wiley-Interscience (1988, including all updates until present), Ed Harlow and David Lane (editors) Antibodies: A Laboratory Manual, Cold Spring Harbour Laboratory, (1988), and J. E. Coligan et al. (editors) Current Protocols in Immunology, John Wiley & Sons (including all updates until present).

The term “and/or”, e.g., “X and/or Y” shall be understood to mean either “X and Y” or “X or Y” and shall be taken to provide explicit support for both meanings or for either meaning.

As used herein, the term “about”, unless stated to the contrary, refers to +/−10%, more preferably +/−5%, of the designated value.

The terms “level” and “amount” are used to define the amount of a particular substance in a sample from a subject or in a cell culture media (or sample therefrom). For example, a particular concentration, weight, percentage (e.g. v/v %) or ratio can be used to define the level of a particular substance in a sample. In an example, the level is expressed in terms of how much of a particular marker is expressed by cells of the disclosure under culture conditions. In an example, expression represents cell surface expression. In another example, the level is expressed in terms of how much of a particular marker is release from cells described herein under culture conditions. In an example, the level of a particular marker is determined in a sample obtained from a patient or subject (e.g. a blood sample or a serum sample).

In an example, the level is expressed in pg/ml. For example, the level of ST2 and/or Reg3α can be expressed in pg/mL. In another example, the level is expressed in ng/mL. In another example, the level is expressed in ng/L. In another example, the level is expressed in pg/L. In another example, the level is expressed as a relative unit based on the level of a standard marker in the sample. Expressing a particular level as a relative unit may make it easier to compare the level with an appropriate control. As would appreciated by those of skill in the art, in the context of MAP, the level of expression is determined so as to allow the resulting value to be used in the formula disclosed herein for determining MAP.

In an example, the level of a particular marker is determined in a sample taken from a subject. For example, the level of ST2, Reg3α or an inflammatory biomarker disclosed herein is determined in a blood sample. In an example, the sample is a serum sample. There are various assays available for measuring Reg3α, ST2 and/or other inflammatory biomarker levels in a sample such as antibody based immunoassays. For example, Reg3α and ST2 levels can be measured in a sample using an Enzyme-Linked Immunosorbent (ELISA) assay. In an example, a blood or serum sample is obtained from a subject and then purified before being contacted with anti-Reg3α and anti-ST2 antibody. In this example, extent of antibody binding is used to quantify the level of Reg3α and ST2 in the sample (e.g. pg/mL). In an example, levels of Reg3α and ST2 are measured in serum. In these examples, the levels of Reg3α and ST2 are used to determine a subject's MAP. In an example, multiple samples are obtained from a subject over time. Reg3α and ST2 levels can be determined in these samples along with the corresponding MAP to monitor a subject's MAP over time. In an example, a sample is taken at baseline (i.e. before administering cell therapy) and post administration of cell therapy. MAP can be compared between samples to determine whether MAP has reduced. In another example, MAP can be determined in multiple samples taken over time (e.g. baseline, day 28, day 100). In these examples, samples can be assessed to determine whether MAP has reduced and, whether a reduction in MAP is durable. In an example, a durable reduction in MAP is determined based on an observed reduction in MAP from baseline in at least two samples post administration of cell therapy. In an example, the samples are taken at day 28 and day 100. In another example, the samples are taken at day 100 and day 160. In another example, the samples are taken at day 160 and day 180. In another example, the samples are taken at day 28, day 100, day 160 and day 180. In an example, the above referenced methods are used to determine the level of one or more of inflammatory biomarkers such as ELAFIN, serum IL-2ra (s IL-2ra), TNFR1, IL-8 and HGF.

Culture expanding cells from a cryopreserved intermediate means thawing cells subject to cryogenic freezing and in vitro culturing under conditions suitable for growth of the cells.

In an example, the present disclosure encompasses selecting certain subjects with GvHD for treatment with a MLPSC composition disclosed herein. In an example, subjects with severe GvHD are selected for treatment. In an example, methods of selecting a subject for treatment comprises the steps of i) determining or having determined a subject's MAP; and ii) selecting a subject having MAP indicative of severe GvHD for treatment. In an example, subjects with an MAP of ≥0.29 are selected for treatment.

In an example, the subject has a reduced risk of mortality after treatment. In one example, the reduced risk may be relative to risk of mortality in a subject that has not been administered MLPSCs. In another example, the reduced risk is relative to the risk of mortality in the same subject before being administered MLPSCs. In these examples, risk is determined according to the subject's MAP. For example, a subject with a MAP of ≥0.29 has a high risk of mortality. In another example, a MAP of <0.29 has a reduced risk of mortality.

In an example, mortality is “non-relapse mortality”. The term “non-relapse mortality” refers to death without recurrent or progressive disease. In an example, the subject has a reduced risk of 6 month non-relapse mortality. In another example, the subject has a reduced risk of 1 year non-relapse mortality.

In an example, treatment increases subject survival. In an example, treatment increases the probability of a subject surviving for at least 100 days after initiation of treatment. In another example, treatment increases the probability of a subject surviving for at least 180 days after initiation of treatment. In an example, the increased probability is determined relative to a subject that is not treated with a composition of the disclosure. In an example, the increased probability is determined relative to a subject that has a MAP of ≥0.29.

The term “subject” as used herein refers to a human subject. For example, the subject can be an adult. In another example, the subject can be a child. In another example, the subject can be an adolescent. In an example, the subject can be a paediatric subject. Paediatric is the branch of medicine that involves the medical care of infants, children, and adolescents. In one example, the subject is less than 25 years of age. In another example, the subject is less than 21 years of age. In another example, the subject is less than 18 years of age. In an example, the paediatric subject can range in age from birth to 17 years old.

Terms such as “subject”, “patient” or “individual” are terms that can, in context, be used interchangeably in the present disclosure.

The term “clinically proven” (used independently or to modify the term “effective”) shall mean that efficacy has been proven by a clinical trial wherein the clinical trial has met the approval standards of U.S. Food and Drug Administration, EMEA or a corresponding national regulatory agency. For example, the clinical study may be an adequately sized, randomized, double-blinded study used to clinically prove the effects of the composition. In an example, a clinically proven effective amount is an amount shown by a clinical trial to meet a specified endpoint. In an example, the end point is protection against death. Put another way, the end point increases survival. For example, 100 day survival may be increased when administering treatment according to the present disclosure.

Accordingly, the terms “clinically proven efficacy” and “clinically proven effective” can be used in the context of the present disclosure to refer to a dose, dosage regimen, treatment or method disclosed herein. Efficacy can be measured based on change in the course of the disease in response to administering a composition disclosed herein. For example, a composition of the disclosure is administered to a subject in an amount and for a time sufficient to induce an improvement, preferably a sustained improvement, in at least one indicator that reflects the severity of GvHD. Various indicators that reflect the severity of the disease can be assessed for determining whether the amount and time of the treatment is sufficient. Such indicators include, for example, clinically recognized indicators of disease severity or symptoms. In an example, the degree of improvement is determined by a physician, who can make this determination based on signs, symptoms, or other test results (e.g. MAP; Reg3α and ST2 levels; skin % BSA; mouth score; eye score of at least one point; skin features score; gastrointestinal tract score; liver score; lung symptom score; lung FEV1 score; joints and fascia score; and/or genital tract score).

In an example, a clinically proven effective amount improves patient survival. In another example, a clinically proven effective amount reduces a subjects risk of mortality, for example non-relapse mortality. In another example, a clinically proven effective amount increases 100 day survival. In an example, methods of the disclosure administer a clinically proven effective amount of a composition disclosed herein.

In an example, compositions of the disclosure comprise genetically unmodified mesenchymal precursor lineage or stem cells. As used herein, the term “genetically unmodified” refers to cells that have not been modified by transfection with a nucleic acid. For the avoidance of doubt, in the context of the present disclosure a mesenchymal lineage precursor or stem cell transfected with a nucleic acid encoding a protein would be considered genetically modified.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or group of compositions of matter.

Those skilled in the art will appreciate that the disclosure described herein is susceptible to variations and modifications other than those specifically described. It is to be understood that the disclosure includes all such variations and modifications. The disclosure also includes all of the steps, features, compositions and compounds referred to or indicated in this specification, individually or collectively, and any and all combinations or any two or more of said steps or features.

The present disclosure is not to be limited in scope by the specific embodiments described herein, which are intended for the purpose of exemplification only. Functionally-equivalent products, compositions and methods are clearly within the scope of the disclosure, as described herein.

Any example disclosed herein shall be taken to apply mutatis mutandis to any other example unless specifically stated otherwise.

### Graft Versus Host Disease

Graft versus Host Disease (GvHD) is an immunological disorder that is the major factor that limits the success and availability of allogeneic bone marrow or stem cell transplantation. GvHD occurs in acute (aGvHD) or chronic (cGvHD) forms. Acute GvHD usually manifests within 100 days following bone marrow or stem cell transplantation. Chronic GvHD generally manifests later than aGvHD (>100 days post transplantation) and has some features of autoimmune diseases. It may develop either de novo, following resolution of aGvHD or as an extension of aGvHD. Chronic GvHD can cause multiple, often debilitating symptoms, including widespread skin rashes, painful mouth ulcers, shortness of breath, and limb and joint pain.

Methods of the present disclosure are effective in a subset of patients with GvHD. These subjects have severe disease. In an example, severe GvHD is acute GvHD. In another example, severe GvHD is chronic GvHD. In an example, severe GvHD presents in the stomach and/or gut of the subject as damage to these organs. In this example, various sites of inflammation can occur along the stomach and/or gut and, in certain examples, it may be preferable to administer treatment directly to one or more of these sites of inflammation. In an example, severe GvHD is accompanied by inflammatory bowel disease (IBD). For example, the GvHD may be accompanied by Crohn's disease.

In an example, severe GvHD is determined based on the subject's MAGIC Algorithm Probability (MAP). MAP is a validated analysis that combines the serum concentrations of two biomarkers, Regenerating islet-derived protein 3 alpha (Reg3a) and soluble interleukin 1 receptor-like 1 (ST2), into a single value that predicts long-term outcomes such as response to therapy after 28 days and 6-month non-relapse mortality (NRM). MAP is calculated by:

$$log 10 [ - log 10 ( 1 - \hat{p} ) ] = - 11.263 + 1.844 ( log 10 ST 2 ) + 0.577 ( log 10 REG 3 α ) ,$$

where \hat{p} is the predicted probability of 6-month NRM (Hartwell et al.; Major-Monfried et al.). Accordingly, in an example, the present disclosure relates to a method for treating GvHD in a subject, the method comprising administering to the subject a composition comprising MLPSCs, wherein the subject has severe GvHD as determined by MAGIC Algorithm Probability (MAP). In another example, subjects are selected for therapy based on their MAP indicating that they have severe GvHD. Thus, in an example, the present disclosure also encompasses a method of selecting GvHD patients for treatment with MLPSCs, the method comprising i) determining or having determined a subject's MAP ii) selecting a subject having MAP indicative of severe GvHD for treatment, preferably, wherein the treatment comprises administering a composition comprising MLPSCs.

More severe GvHD is associated with higher MAP scores. Accordingly, in an example, “severe GvHD” according to the present disclosure is defined based on MAP. In an example, the subject has a MAP greater than 0.16, preferably greater than 0.2, more preferably greater than 0.25. A MAP score of ≥0.291 is a validated threshold that identifies GvHD patients with the most severe disease (Hartwell et al.; Major-Monfried et al.) For example, patients with a MAP score of ≥0.291 are at high risk of non-response to treatment and death. Accordingly, in an example, the subject has a MAP ≥0.29. In an example, the subject has a MAP ≥0.291. In another example, subject has a MAP between 0.15 and 0.8. In another example, the subject has a MAP between 0.16 and 0.7. In another example, subject has a MAP between 0.2 and 0.7. In another example, subject has a MAP between 0.29 and 0.6.

In one example, subjects with severe GvHD as determined by high MAP have a low probability of achieving a clinical response to primary therapy within 28 days and/or a high risk of 6 month non-relapse mortality. Accordingly, in one example, the subject has a 6 month non-relapse mortality risk of ≥70%. In another example, the subject has a 6 month non-relapse mortality risk of ≥80%. In another example, the subject has a 6 month non-relapse mortality risk of ≥90%. In these examples, the subject can have a MAP ≥0.29.

High MAP is indicative of severe gastrointestinal (GI) crypt damage. GI crypts are glands found in between villi in the intestinal epithelium lining of the small intestine and large intestine. GI crypt cells provide stem cells for renewal of the intestinal epithelium. For example, subjects with a MAP of ≥0.29 generally have severe GI crypt damage. In an example, crypt damage is determined by histological assessment. For example, a biopsy of gastrointestinal tissue is taken during an endoscopy or colonoscopy which is then histologically assessed for cellular damage and inflammation. Accordingly, in an example, subjects treated according to the present disclosure can have severe GI crypt damage.

As outlined above, MAP is determined based on Reg3α and ST2 levels. Accordingly, in an example, serum concentrations of Reg3α and ST2 are determined in a subject. In an example, the level of ST2 and Reg3α are quantified in the same sample. In another example, the level of ST2 and Reg3α are quantified in separate samples. In an example, multiple samples are obtained periodically, wherein ST2 and Reg3α are quantified in each sample so that a subject's MAP can be monitored over time.

GvHD severity can also be graded by patterns of organ involvement and clinical performance status. Multi-organ involvement includes skin rash, liver involvement, and/or gastrointestinal (GI) involvement. Examples of skin rash, liver involvement, and GI involvement are provided in Table 1 and Table 2. In an example, the subject has GvHD with multi-organ involvement. In an example, severe GvHD is graded according to the Glucksberg scale (Glucksberg et al, 1974; Thomas et al, 1975) (Table 1). For example, the subject can have Grade II GvHD or Grade III/IV GvHD according to the Glucksberg scale. In one example, the subject has Grade II GvHD. In another example, the subject has Grade III/IV GvHD.

In another example, severe GvHD is graded according to IBMTR Severity Index (Table 2) (Rowlings et al., 1997). In one example, the subject has Grade B, Grade C, or Grade D GvHD according to the IBMTR severity scale.

In another example, the subject has Minnesota high risk GvHD. Minnesota high risk acute GvHD is defined as either skin stage 4; lower GI stage 3-4 or liver stage 3-4; or skin stage 3+ and either lower GI 2-4 or liver stage 2-4 GvHD (MacMillan et al., 2015). In each of these examples, the subject can also have a high MAP score. For example, the subject can have a MAP score ≥0.29.

TABLE 1
Glucksberg clinical stage and grade of
acute GvHD (Rowlings et al., 1997).

| Stage | Skin | Liver | Intestinal tract |
|---|---|---|---|
| 1 | Maculopapular rash | Bilirubin 34-50 | >500 ml |
| | <25% of body | μmol/l | diarrhoea/d |
| | surface | | |
| 2 | Maculopapular rash | Bilirubin 51-102 | >1000 ml |
| | 25-50% of body | μmol/l | diarrhoea/d |
| | surface | | |
| 3 | Generalised | Bilirubin 103-225 | >1500 ml |
| | erythroderma | μmol/l | diarrhoea/d |
| 4 | Generalised | Bilirubin >225 | Severe abdominal |
| | erythroderma with | μmol/l | pain, with or |
| | bullous formation | | without ileus |
| | and desquamation | | |

| Grade | Degree of organ involvement |
|---|---|
| I | Stage 1-2 skin rash: no gut involvement: no liver involvement: |
| | no decrease in clinical performance |
| II | Stage 1-3 skin rash: stage 1 gut involvement or stage 1 liver |
| | involvement (or both): mild decrease in clinical performance |
| III | Stage 2-3 skin rash: stage 2-3 gut involvement or stage 2-4 liver |
| | involvement (or both): marked decrease in clinical performance |
| IV | Similar to Grade III with stage-4 organ involvement |
| | and extreme decrease in clinical performance |

TABLE 2
Criteria for IBMTR Severity Index for
acute GvHD (Rowlings et al., 1997).

| | Skin | | | Liver | | | Gastrointestinal | |
|---|---|---|---|---|---|---|---|---|
| | involvement | | | involvement | | | invovlement | |
| | | Extent | | | Total | | | Volume of |
| In- | Stage | of | | Stage | bilirubin | | Stage | diarrhoea |
| dex* | (max) | rash | | (max) | (μmol/l) | | (max) | (ml/d) |
| A | 1 | <25% | | 0 | <34 | | 0 | <500 |
| B | 2 | 25-50% | or | 1-2 | 34-102 | or | 1-2 | 550-1500 |
| C | 3 | >50% | or | 3 | 103-255 | or | 3 | >1500 |
| D | 4 | Bullae | or | 4 | >255 | or | 4 | Severe |
| | | | | | | | | pain and |
| | | | | | | | | ileus |

*Assign Index based on maximum in an individual organ system

In an example, subjects with severe GvHD do not respond to treatment with primary therapy. In an example, the subject has worsened within 3 days of primary therapy. In this example, the subject has worsened if their GvHD has increased in severity. For example, the subject's GvHD severity has increased according to MAP. In another example, the subject's GvHD severity has increased according to the Glucksberg scale. In another example, the subject's GvHD severity has increased according to the IMBTR scale. In another example, the subject's GvHD severity has increased according to organ involvement.

In another example, the subject has not responded within 7 days of a primary therapy. For example, the subject is refractory to primary therapy. In one example, primary therapy is systemic steroids. In one example, the subject has severe GvHD and is refractory to a steroid. In an example, the steroid is a corticosteroid. In another example, the steroid is a glucocorticoid. In another example, the steroid is prednisone. In another example, the subject has severe GvHD and is refractory to steroids and a second line therapy. For example, a second line therapy can include extracorporeal photopheresis, etanercept, infliximab, ruxolitinib, anti-thymocyte globulin, mycophenolate, alemtuzumab, basiliximab, or tocilizumab.

### Selecting a Patient

In an example, the present disclosure encompasses selecting certain subjects with GvHD for treatment with MLPSCs. In an example, subjects with severe GvHD are selected for treatment. For example, methods of selecting a subject for treatment comprises the steps of i) determining or having determined a subject's MAP; and ii) selecting a subject having MAP indicative of severe GvHD for treatment. In an example, subjects with a MAP of ≥0.29 are selected for treatment. In an example, the method comprises the steps of: i) determining or having determined the subject's Reg3α and ST2 levels; ii) calculating or having calculated the subject's MAP according to the subject's Reg3α and ST2 levels; iii) selecting a subject for treatment with MLPSCs, wherein the subject's MAP is ≥0.29; and iv) administering to the subject a composition comprising MLPSCs. In another example, subjects with a MAP of ≥0.291 are selected for treatment.

### Treatment Response

Methods of the present disclosure relate to the treatment of GvHD. As used herein, the terms “treating”, “treat”, “treatment”, “reducing progression” include administering a population of MLPSCs and/or progeny thereof and/or soluble factors derived therefrom and/or extracellular vesicles derived therefrom to thereby reduce or eliminate at least one symptom of GvHD.

In an example, treatment reduces a subject's ST2 and/or Reg3α level. For example, a subject's ST2 and/or Reg3α level is reduced relative to the subject's baseline level. In an example, treatment reduces a subject's ST2 level. For example, the subject's ST2 level is reduced relative to the subject's baseline ST2 level. In another example, treatment reduces a subject's Reg3α level. For example, the subject's Reg3a level is reduced relative to the subject's baseline Reg3α level. In another example, treatment reduces a subject's MAP. For example, the subject's MAP is reduced relative to the subject's baseline level. In an example, the subject's MAP is reduced to <0.29. In another example, the subject's MAP is reduced to <0.291. In these examples, treatment decreases the subject's MAP by day 28. In another example, treatment decreases the subject's MAP by day 100. In another example, treatment decreases the subject's MAP by day 160. In another example, treatment decreases the subject's MAP by day 180. In an example, the decrease in MAP is sustained for at least 1 month. In another example, the decrease in MAP is sustained for at least 3 months.

In an example, treatment decreases the level of one or more inflammatory biomarkers such as ELAFIN, sIL-2ra, TNFR1, IL-8 and HGF. For example, treatment can reduce the level of ELAFIN. In another example, treatment can reduce the level of sIL-2ra. In another example, treatment can reduce the level of TNFR1. In another example, treatment can reduce the level of IL-8. In example, the level of inflammatory biomarker is reduced between day 50 and 200 after treatment. In an example, the level of inflammatory biomarker is reduced between day 100 and 200 after treatment. In another example, the level of inflammatory biomarker is reduced between by day 100 after treatment. In another example, the level of inflammatory biomarker is reduced between by day 200 after treatment.

In another example, the reduction in the subject's inflammatory biomarker is sustained at day 100, at day 160, at day 180. For example, a reduction in one or more of ELAFIN, sIL-2ra, TNFR1, IL-8 and HGF can be observed at day 100 and sustained at day 180.

In an example, a decreases in the level of one or more of ELAFIN, sIL-2ra, TNFR1, IL-8 and HGF accompanies a decrease in the subjects MAP score.

In an example, the level of one or more inflammatory biomarkers is decreased to level(s) corresponding to subjects with MAP <0.29.

In an example, treatment reduces GI crypt damage in a subject. For example, a subject's GI crypt damage is reduced relative to the subject's baseline level. In these examples, a reduced MAP is indicative of reduced crypt damage. In another example, reduced crypt damage is determined by histological assessment of a gastrointestinal tissue biopsy obtained by endoscopy or colonoscopy. For example, treatment reduces histological signs of cellular damage and inflammation in gastrointestinal crypts.

In an example, treatment is observed at day 28.

As used herein, the term “response” means response to therapy. In an example, a subject is considered to have had a response if they have an improvement in at least one organ without progression in any other organs and if additional therapy was not required. In another example, a subject is considered not to have had a response if they had stable or progressive GvHD or if the subsequent addition of secondary therapy is required. In this example, a subject who does not have a response is a non-responder.

In an example, treatment induces a partial response. In an example, the partial response is induced at least 28 after treatment is initiated. In an example, the partial response is induced 28 days after treatment is initiated. In an example, the partial response is induced at least 30 days after treatment is initiated. In an example, the partial response is induced at least 2 months after treatment is initiated. In another example, the partial response is induced at least 3 months after treatment is initiated. In another example, the partial response is induced within 3 months. In another example, the partial response is induced 28 to 56 days after treatment is initiated. In another example, the partial response is induced 100 days after treatment is initiated. In another example, the partial response is induced 160 days after treatment is initiated. In another example, the partial response is induced 180 days after treatment is initiated.

In another example, the partial response is induced after two doses. In another example, the partial response is induced after two doses administered once weekly. In another example, the partial response is induced after two doses administered once weekly every two weeks. In another example, the partial response is induced after three doses or more. In an example, a partial response is characterized by one or more or all of:
Reduction in Skin % BSA score of at least one point;
Reduction in mouth score of at least one point;
Reduction in eye score of at least one point;
Reduction in skin features score of at least one point;
Reduction in gastrointestinal tract score of at least one point;
Reduction in liver score of at least one point;
Reduction in lung symptom score of at least one point;
Reduction in lung FEV1 score of at least one point;
Reduction in joints and fascia score of at least one point;
Reduction in genital tract score of at least one point.

In an example, a partial response is characterized by a reduction in Skin % BSA score of at least one point. In another example, a partial response is characterized by a reduction in mouth score of at least one point. In another example, a partial response is characterized by a reduction in eye score of at least one point. In these examples, scores can be obtained using the NIH Consensus Criteria 2014 for GvHD.

In another example, a partial response is characterized by one or more or all of:
Reduction in Skin % BSA score of at least one point;
Reduction in mouth score of at least one point;
Reduction in eye score of at least one point.

There are various classification systems for characterizing GvHD (Lee, S., (2017) Blood., 129(1): 30-37). In an example, the NIH Consensus Criteria 2014 can be used for scoring outcomes disclosed herein (Jagasia et al., (2015) Biol Blood Marrow Transplant., 21:389-401). The components of the NIH Consensus Criteria are shown in the following table:

TABLE 3
Organ Scoring of GvHD

| Organ | Score 0 | Score 1 | Score 2 | Score 3 |
|---|---|---|---|---|
| Skin % | No BSA | 1-18% | 19-50% | >50% |
| BSA¹ | involved | BSA | BSA | BSA |
| Skin | No | N/A | Superficial | Deep sclerotic |
| Features | sclerotic | | sclerotic | features; |
| | features | | features, | “hidebound”; |
| | | | but not | impaired |
| | | | “hidebound” | mobility; |
| | | | | ulceration |
| Mouth | No | Mild | Moderate | Severe |
| | symptoms | symptoms | symptoms with | symptoms with |
| | | with | disease signs | disease signs |
| | | disease | with partial | with major |
| | | signs | limitation | limitation |
| | | but not | of oral intake | of oral |
| | | limiting | | intake |
| | | intake | | |
| | | significantly | | |
| Eyes | No | Mild dry | Moderate dry | Severe dry eye |
| | symptoms | eye | eye symptoms | symptoms |
| | | symptoms | partially | significantly |
| | | not | affecting ADL | affecting ADL |
| | | affecting | (requiring | (special |
| | | ADL | lubricant drops | eyewear to |
| | | (requirement | >3x/day or | relieve pain) OR |
| | | of lubricant | punctal plugs) | unable to work |
| | | drops | WITHOUT | because of ocular |
| | | ≤3x/day) | new vision | symptoms OR |
| | | | impairment | loss of vision |
| | | | due to | due to kerato- |
| | | | kerato- | conjunctivitis |
| | | | conjunctivitis | sicca (KCS) |
| | | | sicca (KCS) | |
| GI Tract | No | Symptoms | Symptoms | Symptoms |
| | symptoms | without | associated | associated with |
| | | significant | with mild to | significant |
| | | weight | moderate | weight |
| | | loss | weight loss (5- | loss (>15%) |
| | | (<5%) | 15%) within 3 | within 3 |
| | | | months OR | months, requires |
| | | | moderate | nutritional |
| | | | diarrhea | supplement for |
| | | | without | most |
| | | | significant | calorie needs |
| | | | interference | OR esophageal |
| | | | with | dilation |
| | | | daily living | OR severe |
| | | | | diarrhea with |
| | | | | significant |
| | | | | interference with |
| | | | | daily living |
| Liver | Normal | Normal total | Elevated total | Elevated total |
| | total | bilirubin | bilirubin but ≤3 | bilirubin but >3 |
| | bilirubin | with ALT | mg/dL or ALT | mg/dL |
| | and ALT | ≥3 to 5 x | >5 x ULN | |
| | or AP <3 | ULN or AP | | |
| | x ULN | ≥3 x ULN | | |
| Lungs | No | Mild | Moderate | Severe symptoms |
| Symptom | symptoms | symptoms | symptoms (SOB | (SOB at rests; |
| Score | | (SOB after | after walking on | requires 02) |
| | | climbing | flat ground) | |
| | | one flight | | |
| | | of steps) | | |
| Lungs | FEV1 | FEV1 | FEV1 40-59% | FEV1 ≤39% |
| Lung | ≥80% | 60-79% | | |
| Score | | | | |
| Joints | No | Mild | Tightness of | Contractures |
| and | symptoms | tightness | arms or | WITH s |
| Fascia | | of | legs OR joint | ignificant |
| | | arms and | contractures, | decrease of range |
| | | legs, | erythema | of motion AND |
| | | normal | thought to | significant |
| | | or mild | be due to | limitation of |
| | | decreased | fasciitis, | ADL (unable |
| | | range of | moderate | to tie shoes, |
| | | motion | decrease | button shirts, |
| | | AND not | of range | dress shelf, etc.) |
| | | affecting | of motion | |
| | | ADL | AND mild | |
| | | | to moderate | |
| | | | limitation | |
| | | | of ADL | |
| Genital | No signs | Mild signs | Moderate signs | Severe |
| Tract² | | and females | and may | signs with |
| | | with or | have signs of | or without |
| | | without | discomfort on | symptoms |
| | | discomfort | exam | |
| | | on exam | | |
| Other | No | Mild | Moderate | Severe |
| features³ | GvHD | | | |

In an example, a partial response is a decrease of ≥1 point on the organ-specific NIH Consensus Criteria 2014 score from the Table above. Accordingly, in an example, treatment induces ≥1 point decrease in Skin % BSA score. In another example, treatment induces ≥1 point decrease in mouth score. In another example, treatment induces ≥1 point decrease in eye score. In another example, treatment induces ≥1 point decrease in skin features score. In another example, treatment induces ≥1 point decrease in gastrointestinal tract score. In another example, treatment induces ≥1 point decrease in liver score. In another example, treatment induces ≥1 point decrease in lung symptom score. In another example, treatment induces ≥1 point decrease in lung FEV1 score. In another example, treatment induces ≥1 point decrease in joints and fascia score. In another example, treatment induces ≥1 point decrease in genital tract score.

In an example, the treatment induces a complete response after treatment is initiated. In an example, a complete response is the complete resolution of GvHD symptoms in all organs. In an example, the complete response is induced 28 days after treatment is initiated. In an example, the complete response is induced at least 28 after treatment is initiated. In an example, the complete response is induced at least 30 after treatment is initiated. In an example, the complete response is induced at least 2 months after treatment is initiated. In another example, the complete response is induced at least 3 months after treatment is initiated. In another example, the complete response is induced 28 to 56 days after treatment is initiated. In another example, the complete response is induced 100 days after treatment is initiated. In another example, the complete response is induced 160 days after treatment is initiated. In another example, the complete response is induced 180 days after treatment is initiated.

In another example, the complete response is induced after two doses. In another example, the complete response is induced after two doses administered once weekly. In another example, the complete response is induced after two doses administered once weekly every two weeks. In another example, the complete response is induced after three doses or more.

In another example, the treatment increases the probability of the subject's survival. For example, treatment increases the probability of the subject surviving for at least 20 days to 200 days after initiation of treatment. In one example, treatment increases the probability of the subject surviving for at least 180 days after initiation of treatment. In another example, treatment increases the probability of the subject surviving at least 100 days. In an example, the increased probability is determined relative to a subject that is not treated with a composition of the disclosure. In an example, the increased probability is determined relative to a subject that has a MAP of ≥0.29.

In another example, the treatment decreases the subject's risk of 6 month non-relapse mortality. For example, the subject's risk of 6 month non-relapse mortality to between 20% and 80%. In one example, the subject's risk is decreased to at least 70%. In another example, the subject's risk is decreased to at least 60%. In another example, the subject's risk is decreased to at least 50%. In another example, the subject's risk is decreased to at least 40%. In another example, the subject's risk is decreased to at least 30%. In another example, the treatment decreases the subject's risk of 1 year non-relapse mortality. For example, the subject's risk of 1 year non-relapse mortality is decreased to between 20% and 80%. In one example, the subject's risk is decreased to at least 70%. In another example, the subject's risk is decreased to at least 60%. In another example, the subject's risk is decreased to at least 50%. In another example, the subject's risk is decreased to at least 40%. In another example, the subject's risk is decreased to at least 30%.

### Mesenchymal Lineage Precursor or Stem Cells

As used herein, the term “mesenchymal lineage precursor or stem cell (MLPSC)” refers to undifferentiated multipotent cells that have the capacity to self-renew while maintaining multipotency and the capacity to differentiate into a number of cell types either of mesenchymal origin, for example, osteoblasts, chondrocytes, adipocytes, stromal cells, fibroblasts and tendons, or non-mesodermal origin, for example, hepatocytes, neural cells and epithelial cells. For the avoidance of doubt, a “mesenchymal lineage precursor cell” refers to a cell which can differentiate into a mesenchymal cell such as bone, cartilage, muscle and fat cells, and fibrous connective tissue.

The term “mesenchymal lineage precursor or stem cells” includes both parent cells and their undifferentiated progeny. The term also includes mesenchymal precursor cells (MPCs), multipotent stromal cells, mesenchymal stem cells (MSCs), perivascular mesenchymal precursor cells, and their undifferentiated progeny.

MLPSCs can be autologous, allogeneic, xenogenic, syngenic or isogenic. Autologous cells are isolated from the same individual to which they will be reimplanted. Allogeneic cells are isolated from a donor of the same species. Xenogenic cells are isolated from a donor of another species. Syngenic or isogenic cells are isolated from genetically identical organisms, such as twins, clones, or highly inbred research animal models.

In an example, the MLPSCs are allogeneic. In an example, the allogeneic MLPSCs are culture expanded and cryopreserved.

MLPSCs reside primarily in the bone marrow, but have also shown to be present in diverse host tissues including, for example, cord blood and umbilical cord, adult peripheral blood, adipose tissue, trabecular bone and dental pulp. They are also found in skin, spleen, pancreas, brain, kidney, liver, heart, retina, brain, hair follicles, intestine, lung, lymph node, thymus, ligament, tendon, skeletal muscle, dermis, and periosteum; and are capable of differentiating into germ lines such as mesoderm and/or endoderm and/or ectoderm. Thus, MLPSCs are capable of differentiating into a large number of cell types including, but not limited to, adipose, osseous, cartilaginous, elastic, muscular, and fibrous connective tissues. The specific lineage-commitment and differentiation pathway which these cells enter depends upon various influences from mechanical influences and/or endogenous bioactive factors, such as growth factors, cytokines, and/or local microenvironmental conditions established by host tissues.

The terms “enriched”, “enrichment” or variations thereof are used herein to describe a population of cells in which the proportion of one particular cell type or the proportion of a number of particular cell types is increased when compared with an untreated population of the cells (e.g., cells in their native environment). In one example, a population enriched for MLPSCs comprises at least about 0.1% or 0.5% or 1% or 2% or 5% or 10% or 15% or 20% or 25% or 30% or 50% or 75% MLPSCs. In this regard, the term “population of cells enriched for MLPSCs” will be taken to provide explicit support for the term “population of cells comprising X % MLPSCs”, wherein X % is a percentage as recited herein. The MLPSCs can, in some examples, form clonogenic colonies, e.g. CFU-F (fibroblasts) or a subset thereof (e.g., 50% or 60% or 70% or 70% or 90% or 95%) can have this activity.

In an example of the present disclosure, the MLPSCs are mesenchymal stem cells (MSCs). The MSCs may be a homogeneous composition or may be a mixed cell population enriched in MSCs. Homogeneous MSC compositions may be obtained by culturing adherent marrow or periosteal cells, and the MSCs may be identified by specific cell surface markers which are identified with unique monoclonal antibodies. A method for obtaining a cell population enriched in MSCs is described, for example, in U.S. Pat. No. 5,486,359. Alternative sources for MSCs include, but are not limited to, blood, skin, cord blood, muscle, fat, bone, and perichondrium. In an example, the MSCs are allogeneic. In an example, the MSCs are cryopreserved. In an example, the MSCs are culture expanded and cryopreserved.

In another example, the MLPSCs are CD29+, CD54+, CD73+, CD90+, CD102+, CD105+, CD106+, CD166+, MHC1+MSCs.

Isolated or enriched MLPSCs can be expanded in vitro by culture. Isolated or enriched MLPSCs can be cryopreserved, thawed and subsequently expanded in vitro by culture.

In one example, isolated or enriched MLPSCs are seeded at 50,000 viable cells/cm² in culture medium (serum free or serum-supplemented), for example, alpha minimum essential media (αMEM) supplemented with 5% fetal bovine serum (FBS) and glutamine, and allowed to adhere to the culture vessel overnight at 37° C., 20% O₂. The culture medium is subsequently replaced and/or altered as required and the cells cultured for a further 68 to 72 hours at 37° C., 5% O₂.

As will be appreciated by those of skill in the art, cultured MLPSCs are phenotypically different to cells in vivo. For example, in one embodiment they express one or more of the following markers, CD44, NG2, DC146 and CD140b. Cultured MLPSCs are also biologically different to cells in vivo, having a higher rate of proliferation compared to the largely non-cycling (quiescent) cells in vivo.

In one example, the population of cells is enriched from a cell preparation comprising STRO-1+ cells in a selectable form. In this regard, the term “selectable form” will be understood to mean that the cells express a marker (e.g., a cell surface marker) permitting selection of the STRO-1+ cells. The marker can be STRO-1, but need not be. For example, as described and/or exemplified herein, cells (e.g., mesenchymal precursor cells (MPCs)) expressing STRO-2 and/or STRO-3 (TNAP) and/or STRO-4 and/or VCAM-1 and/or CD146 and/or 3G5 also express STRO-1 (and can be STRO-1bright). Accordingly, an indication that cells are STRO-1+ does not mean that the cells are selected solely by STRO-1 expression. In one example, the cells are selected based on at least STRO-3 expression, e.g., they are STRO-3+ (TNAP+). For example, the MPCs can be isolated from bone mononuclear cells with an anti-STRO-3 antibody.

Reference to selection of a cell or population thereof does not necessarily require selection from a specific tissue source. As described herein STRO-1+ cells can be selected from or isolated from or enriched from a large variety of sources. That said, in some examples, these terms provide support for selection from any tissue comprising STRO-1+ cells (e.g., mesenchymal precursor cells) or vascularized tissue or tissue comprising pericytes (e.g., STRO-1+ pericytes) or any one or more of the tissues recited herein.

In one example, the cells used in the present disclosure express one or more markers individually or collectively selected from the group consisting of TNAP+, VCAM-1+, THY-1+, STRO-2+, STRO-4+ (HSP-90β), CD45+, CD146+, 3G5+ or any combination thereof.

By “individually” is meant that the disclosure encompasses the recited markers or groups of markers separately, and that, notwithstanding that individual markers or groups of markers may not be separately listed herein the accompanying claims may define such marker or groups of markers separately and divisibly from each other.

By “collectively” is meant that the disclosure encompasses any number or combination of the recited markers or groups of markers, and that, notwithstanding that such numbers or combinations of markers or groups of markers may not be specifically listed herein the accompanying claims may define such combinations or sub-combinations separately and divisibly from any other combination of markers or groups of markers.

As used herein the term “TNAP” is intended to encompass all isoforms of tissue non-specific alkaline phosphatase. For example, the term encompasses the liver isoform (LAP), the bone isoform (BAP) and the kidney isoform (KAP). In one example, the TNAP is BAP. In one example, TNAP as used herein refers to a molecule which can bind the STRO-3 antibody produced by the hybridoma cell line deposited with ATCC on 19 Dec. 2005 under the provisions of the Budapest Treaty under deposit accession number PTA-7282.

Furthermore, in one example, the STRO-1+ cells are capable of giving rise to clonogenic CFU-F.

In one example, a significant proportion of the STRO-1+ cells are capable of differentiation into at least two different germ lines. Non-limiting examples of the lineages to which the STRO-1+ cells may be committed include bone precursor cells; hepatocyte progenitors, which are multipotent for bile duct epithelial cells and hepatocytes; neural restricted cells, which can generate glial cell precursors that progress to oligodendrocytes and astrocytes; neuronal precursors that progress to neurons; precursors for cardiac muscle and cardiomyocytes, glucose-responsive insulin secreting pancreatic beta cell lines. Other lineages include, but are not limited to, odontoblasts, dentin-producing cells and chondrocytes, and precursor cells of the following: retinal pigment epithelial cells, fibroblasts, skin cells such as keratinocytes, dendritic cells, hair follicle cells, renal duct epithelial cells, smooth and skeletal muscle cells, testicular progenitors, vascular endothelial cells, tendon, ligament, cartilage, adipocyte, fibroblast, marrow stroma, cardiac muscle, smooth muscle, skeletal muscle, pericyte, vascular, epithelial, glial, neuronal, astrocyte and oligodendrocyte cells.

In an example, MLPSCs are obtained from a single donor, or multiple donors where the donor samples or MLPSCs are subsequently pooled and then culture expanded.

MLPSCs encompassed by the present disclosure may also be cryopreserved prior to administration to a subject. In an example, MLPSCs are culture expanded and cryopreserved prior to administration to a subject.

In an example, the present disclosure encompasses MLPSCs as well as progeny thereof, soluble factors derived therefrom, and/or extracellular vesicles isolated therefrom. In another example, the present disclosure encompasses MLPSCs as well as extracellular vesicles isolated therefrom. For example, it is possible to culture expand MLPSCs of the disclosure for a period of time and under conditions suitable for secretion of extracellular vesicles into the cell culture medium. Secreted extracellular vesicles can subsequently be obtained from the culture medium for use in therapy.

The term “extracellular vesicles” as used herein, refers to lipid particles naturally released from cells and ranging in size from about 30 nm to as a large as 10 microns, although typically they are less than 200 nm in size. They can contain proteins, nucleic acids, lipids, metabolites, or organelles from the releasing cells (e.g., mesenchymal stem cells; STRO-1⁺ cells).

The term “exosomes” as used herein, refers to a type of extracellular vesicle generally ranging in size from about 30 nm to about 150 nm and originating in the endosomal compartment of mammalian cells from which they are trafficked to the cell membrane and released. They may contain nucleic acids (e.g., RNA; microRNAs), proteins, lipids, and metabolites and function in intercellular communication by being secreted from one cell and taken up by other cells to deliver their cargo.

In an example, compositions of the disclosure comprise cells that induce new blood vessel formation in target tissue. In an example, the target tissue is the heart. In another example, the cells secrete factors that protect at risk or damaged myocardium. In an example, at risk or damaged myocardium has been subject to a lack of blood flow resulting from an ischemic event. In an example, the cells secrete factors that reduce apoptosis in cardiomyocytes.

### Culture Expansion of the Cells

In an example, MLPSCs are culture expanded. “Culture expanded” MLPSC media are distinguished from freshly isolated cells in that they have been cultured in cell culture medium and passaged (i.e. sub-cultured). In an example, culture expanded MLPSCs are culture expanded for about 4-10 passages. In an example, MLPSCs are culture expanded for at least 5, at least 6, at least 7, at least 8, at least 9, at least 10 passages. For example, MLPSCs can be culture expanded for at least 5 passages. In an example, MLPSCs can be culture expanded for at least 5-10 passages. In an example, MLPSCs can be culture expanded for at least 5-8 passages. In an example, MLPSCs can be culture expanded for at least 5-7 passages. In an example, MLPSCs can be culture expanded for more than 10 passages. In another example, MLPSCs can be culture expanded for more than 7 passages. In these examples, stem cells may be culture expanded before being cryopreserved to provide an intermediate cryopreserved MLPSC population. In an example, compositions of the present disclosure are produced by culturing cells from an intermediate cryopreserved MLPSC population or, put another way, a cryopreserved intermediate.

In an example, compositions of the disclosure comprise MLPSCs that are culture expanded from a cryopreserved intermediate. In an example, the cells culture expanded from a cryopreserved intermediate are culture expanded for at least 5, at least 6, at least 7, at least 8, at least 9, at least 10 passages. For example, MLPSCs can be culture expanded for at least 5 passages. In an example, MLPSCs can be culture expanded for at least 5-10 passages. In an example, MLPSCs cells can be culture expanded for at least 5-8 passages. In an example, MLPSCs can be culture expanded for at least 5-7 passages. In an example, MLPSCs can be culture expanded for more than 10 passages. In another example, MLPSCs can be culture expanded for more than passages.

In an example, MLPSCs culture expanded from a cryopreserved intermediate can be culture expanded in medium free of animal proteins. In an example, MLPSCs culture expanded from a cryopreserved intermediate can be culture expanded in xeno-free medium. In an example, MLPSCs culture expanded from a cryopreserved intermediate can be culture expanded in medium that is fetal bovine serum free.

In an embodiment, MLPSCs can be obtained from a single donor, or multiple donors where the donor samples or MLPSCs are subsequently pooled and then culture expanded. In an example, the culture expansion process comprises:
i. expanding by passage expansion the number of viable cells to provide a preparation of at least about 1 billion of the viable cells, wherein the passage expansion comprises establishing a primary culture of isolated MLPSCs and then serially establishing a first non-primary (P1) culture of isolated MLPSCs from the previous culture;
ii. expanding by passage expansion the P1 culture of isolated MLPSCs to a second non-primary (P2) culture of MLPSCs; and,
iii. preparing and cryopreserving an in-process intermediate MLPSC preparation obtained from the P2 culture of MLPSCs; and,
iv. thawing the cryopreserved in-process intermediate MLPSC preparation and expanding by passage expansion the in-process intermediate MLPSC preparation.

In an example, the expanded MLPSC preparation has an antigen profile and an activity profile comprising:
i. less than about 0.75% CD45+ cells;
ii. at least about 95% CD105+ cells;
iii. at least about 95% CD166+ cells.

In an example, the expanded MLPSC preparation is capable of inhibiting IL2-Rα expression by CD3/CD28-activated PBMCs by at least about 30% relative to a control. In another example, the expanded MLPSC preparation is capable of inhibiting IL2-Rα expression by CD3/CD28-activated PBMCs by at least about 40% relative to a control. In another example, the expanded MLPSC preparation is capable of inhibiting IL2-Rα expression by CD3/CD28-activated PBMCs by at least about 50% relative to a control. In another example, the expanded MLPSC preparation is capable of inhibiting IL2-Rα expression by CD3/CD28-activated PBMCs by at least about 70% relative to a control. In another example, the expanded MLPSC preparation is capable of inhibiting IL2-Rα expression by CD3/CD28-activated PBMCs by between at least 60% and 80% relative to a control. In another example, the expanded MLPSC preparation is capable of inhibiting IL2-Rα expression by CD3/CD28-activated PBMCs by between at least 70% and 90% relative to a control.

In an example, culture expanded MLPSCs are culture expanded for about 4-10 passages, wherein the MLPSCs have been cryopreserved after at least 2 or 3 passages before being further culture expanded. In an example, MLPSCs are culture expanded for at least 1, at least 2, at least 3, at least 4, at least 5 passages, cryopreserved and then further culture expanded for at least 1, at least 2, at least 3, at least 4, at least 5 passages before being cultured according to the methods of the disclosure.

The process of MLPSC isolation and ex vivo expansion can be performed using any equipment and cell handing methods known in the art. Various culture expansion embodiments of the present disclosure employ steps that require manipulation of cells, for example, steps of seeding, feeding, dissociating an adherent culture, or washing. Any step of manipulating cells has the potential to insult the cells. Although MLPSCs can generally withstand a certain amount of insult during preparation, cells are preferably manipulated by handling procedures and/or equipment that adequately performs the given step(s) while minimizing insult to the cells.

In an example, MLPSCs are washed in an apparatus that includes a cell source bag, a wash solution bag, a recirculation wash bag, a spinning membrane filter having inlet and outlet ports, a filtrate bag, a mixing zone, an end product bag for the washed cells, and appropriate tubing, for example, as described in U.S. Pat. No. 6,251,295, which is hereby incorporated by reference.

In an example, a MLPSC composition cultured according to the present disclosure is 95% homogeneous with respect to being CD105 positive and CD166 positive and being CD45 negative. In an example, this homogeneity persists through ex vivo expansion; i.e. though multiple population doublings.

In an example, MLPSCs of the disclosure are culture expanded in 3D culture. For example, MLPSCs of the disclosure can be culture expanded in a bioreactor. In an example, MLPSCs of the disclosure are initially culture expanded in 2D culture prior to being further expanded in 3D culture. In an example, MLPSCs of the disclosure are culture expanded from a master cell bank. In an example, MLPSCs of the disclosure are culture expanded from a master cell bank in 2D culture before seeding in 3D culture. In an example, MLPSCs of the disclosure are culture expanded from a master cell bank in 2D culture for at least 3 days before seeding in 3D culture in a bioreactor. In an example, MLPSCs of the disclosure are culture expanded from a master cell bank in 2D culture for at least 4 days before seeding in 3D culture in a bioreactor. In an example, MLPSCs of the disclosure are culture expanded from a master cell bank in 2D culture for between 3 and 5 days before seeding in 3D culture in a bioreactor. In these examples, 2D culture can be performed in a cell factory. Various cell factory products are available commercially (e.g. Thermofisher, Sigma).

In an example, cells of the disclosure are STRO-3+ before they are culture expanded.

### Cell Culture Medium

MLPSCs disclosed herein can be culture expanded in various suitable growth mediums.

The term “medium” or “media” as used in the context of the present disclosure, includes the components of the environment surrounding the cells. The media contributes to and/or provides the conditions suitable to allow cells to grow. Media may be solid, liquid, gaseous or a mixture of phases and materials. Media can include liquid growth media as well as liquid media that do not sustain cell growth. Media also include gelatinous media such as agar, agarose, gelatin and collagen matrices. Exemplary gaseous media include the gaseous phase that cells growing on a petri dish or other solid or semisolid support are exposed to.

The cell culture media used for culture expansion contains all essential amino acids and may also contain non-essential amino acids. In general, amino acids are classified into essential amino acids (Thr, Met, Val, Leu, Ile, Phe, Trp, Lys, His) and non-essential amino acids (Gly, Ala, Ser, Cys, Gln, Asn, Asp, Tyr, Arg, Pro).

Those of skill in the art will appreciate that for optimal results, the basal medium must be appropriate for the cell line of interest. For example, it may be necessary to increase the level of glucose (or other energy source) in the basal medium, or to add glucose (or other energy source) during the course of culture, if this energy source is found to be depleted and to thus limit growth. In an example, dissolved oxygen (DO) levels can also be controlled.

In an example, the cell culture medium contains human derived additives. For example, human serum and human platelet cell lysate can be added to the cell culture media.

In an example, the cell culture medium contains only human derived additives. Thus, in an example, the cell culture media is xeno-free. For avoidance of doubt, in these examples, the culture medium is free of animal proteins. In an example, cell culture medium used in the methods of the disclosure is free of animal components.

In an example, the culture medium comprises serum. In other examples the culture medium is fetal bovine serum free culture medium comprising growth factors that promote MLPSCs proliferation. In an embodiment, the culture medium is serum free stem cell culture medium. In an example, the cell culture medium comprises:
a basal medium;
platelet derived growth factor (PDGF);
fibroblast growth factor 2 (FGF2).

In an example, the culture medium comprises platelet derived growth factor (PDGF) and fibroblast growth factor 2 (FGF2), wherein the level of FGF2 is less than about 6 ng/ml. For example, the FGF2 level may be less than about 5 ng/ml, less than about 4 ng/ml, less than about 3 ng/ml, less than about 2 ng/ml, less than about 1 ng/ml. In other examples, the FGF2 level is less than about 0.9 ng/ml, less than about 0.8 ng/ml, less than about 0.7 ng/ml, less than about 0.6 ng/ml, less than about 0.5 ng/ml, less than about 0.4 ng/ml, less than about 0.3 ng/ml, less than about 0.2 ng/ml.

In another example, the level of FGF2 is between about 1 μg/ml and 100 μg/ml. In another example, the level of FGF2 is between about 5 μg/ml and 80 μg/ml. In an example, the level of FGF2 is about 1 ng/ml.

In an example, the PDGF is PDGF-BB. In an example, the level of PDGF-BB is between about 1 ng/ml and 150 ng/ml. In another example, the level of PDGF-BB is between about 7.5 ng/ml and 120 ng/ml. In another example, the level of PDGF-BB is between about 15 ng/ml and 60 ng/ml. In another example, the level of PDGF-BB is at least about 10 ng/ml. In another example, the level of PDGF-BB is at least about 15 ng/ml. In another example, the level of PDGF-BB is at least about 20 ng/ml. In another example, the level of PDGF-BB is at least about 21 ng/ml. In another example, the level of PDGF-BB is at least about 22 ng/ml. In another example, the level of PDGF-BB is at least about 23 ng/ml. In another example, the level of PDGF-BB is at least about 24 ng/ml. In another example, the level of PDGF-BB is at least about 25 ng/ml.

In another example, the PDGF is PDGF-AB. In an example, the level of PDGF-AB is between about 1 ng/ml and 150 ng/ml. In another example, the level of PDGF-AB is between about 7.5 ng/ml and 120 ng/ml. In another example, the level of PDGF-AB is between about 15 ng/ml and 60 ng/ml. In another example, the level of PDGF-AB is at least about 10 ng/ml. In another example, the level of PDGF-AB is at least about 15 ng/ml. In another example, the level of PDGF-AB is at least about 20 ng/ml. In another example, the level of PDGF-AB is at least about 21 ng/ml. In another example, the level of PDGF-AB is at least about 22 ng/ml. In another example, the level of PDGF-AB is at least about 23 ng/ml. In another example, the level of PDGF-AB is at least about 24 ng/ml. In another example, the level of PDGF-AB is at least about 25 ng/ml.

In other examples, additional factors can be added to the cell culture medium. In an example, the culture medium further comprising EGF. EGF is a growth factor that stimulates cell proliferation by binding to its receptor EGFR. In an example, the method of the present disclosure comprises culturing a population of stem cells in a fetal bovine serum free cell culture medium further comprising EGF. In an example, the level of EGF is between about 0.1 and 7 ng/ml. For example, the level of EGF can be at least about 5 ng/ml.

In another example, the level of EGF is between about 0.2 ng/ml and 3.2 ng/ml. In another example, the level of EGF is between about 0.4 ng/ml and 1.6 ng/ml. In another example, the level of EGF is between about 0.2 ng/ml. In another example, the level of EGF is at least about 0.3 ng/ml. In another example, the level of EGF is at least about 0.4 ng/ml. In another example, the level of EGF is at least about 0.5 ng/ml. In another example, the level of EGF is at least about 0.6 ng/ml. In another example, the level of EGF is at least about 0.7 ng/ml. In another example, the level of EGF is at least about 0.8 ng/ml. In another example, the level of EGF is at least about 0.9 ng/ml. In another example, the level of EGF is at least about 1.0 ng/ml.

In the above examples, basal medium such as Alpha MEM or StemSpan™ can be supplemented with the referenced quantity of growth factor. In an example, the culture medium comprises Alpha MEM or StemSpan™ supplemented with 32 ng/ml PDGF-BB, 0.8 ng/ml EGF and 0.02 ng/ml FGF2. In an example, the culture medium comprises Alpha MEM or StemSpan™ supplemented with 10 ng/ml PDGF-BB, 5 ng/ml EGF and 1 ng/ml FGF2.

In other examples, additional factors can be added to the cell culture medium. For example, the cell culture media can be supplemented with one or more stimulatory factors selected from the group consisting of epidermal growth factor (EGF), 1α,25-dihydroxyvitamin D3 (1,25D), tumor necrosis factor α (TNF-α), interleukin-1β (IL-1β) and stromal derived factor 1α (SDF-1α). In another embodiment, cells may also be cultured in the presence of at least one cytokine in an amount adequate to support growth of the cells. In another embodiment, cells can be cultured in the presence of heparin or a derivative thereof. For example, the cell culture medium may contain about 50 ng/ml of heparin. In other examples, the cell culture medium contains about ng/ml of heparin, about 70 ng/ml of heparin, about 80 ng/ml of heparin, about ng/ml of heparin, about 100 ng/ml of heparin, about 110 ng/ml of heparin, about ng/ml of heparin, about 120 ng/ml of heparin, about 130 ng/ml of heparin, about ng/ml of heparin, about 150 ng/ml of heparin or a derivative thereof. In an example, the heparin derivative is a sulphate). Various forms of heparin sulphate are known in the art and include heparin sulphate 2 (HS2). HS2 can be derived from various sources including for example, the liver of male and/or female mammals. Thus, an exemplary heparin sulphate includes male liver heparin sulphate (MML HS) and female liver heparin sulphate (FML HS).

In another example, the cell culture medium of the present disclosure promotes stem cell proliferation while maintaining stem cells in an undifferentiated state. Stem cells are considered to be undifferentiated when they have not committed to a specific differentiation lineage. As discussed above, stem cells display morphological characteristics that distinguish them from differentiated cells. Furthermore, undifferentiated stem cells express genes that may be used as markers to detect differentiation status. The polypeptide products may also be used as markers to detect differentiation status. Accordingly, one of skill in the art could readily determine whether the methods of the present disclosure maintain stem cells in an undifferentiated state using routine morphological, genetic and/or proteomic analysis.

### Modification of the Cells

The MLPSCs disclosed herein may be altered in such a way that upon administration, lysis of the cell is inhibited. Alteration of an antigen can induce immunological non-responsiveness or tolerance, thereby preventing the induction of the effector phases of an immune response (e.g., cytotoxic T cell generation, antibody production etc.) which are ultimately responsible for rejection of foreign cells in a normal immune response. Antigens that can be altered to achieve this goal include, for example, MHC class I antigens, MHC class II antigens, LFA-3 and ICAM-1.

The MLPSCs may also be genetically modified to express proteins of importance for the differentiation and/or maintenance of striated skeletal muscle cells. Exemplary proteins include growth factors (TGF-β, insulin-like growth factor 1 (IGF-1), FGF), myogenic factors (e.g. myoD, myogenin, myogenic factor 5 (Myf5), myogenic regulatory factor (MRF)), transcription factors (e.g. GATA-4), cytokines (e.g. cardiotropin-1), members of the neuregulin family (e.g. neuregulin 1, 2 and 3) and homeobox genes (e.g. Csx, tinman and NKx family).

### Compositions

MLPSCs disclosed herein can be culture expanded from a cryopreserved intermediate to produce a preparation containing at least one therapeutic dose.

In an example, compositions of the disclosure comprise between 10×10⁶ cells and 35×10⁶ cells. In another example, the composition comprises between 20×10⁶ cells and 30×10⁶ cells. In other examples, the composition comprises at least 100×10⁶ cells. In another example, the composition comprises between 50×10⁶ cells and 500×10⁶ cells. In other examples, compositions of the disclosure comprise 150 million cells.

In one example, compositions of the disclose comprise a pharmaceutically acceptable carrier and/or excipient. The terms “carrier” and “excipient” refer to compositions of matter that are conventionally used in the art to facilitate the storage, administration, and/or the biological activity of an active compound (see, e.g., Remington's Pharmaceutical Sciences, 16th Ed., Mac Publishing Company (1980). A carrier may also reduce any undesirable side effects of the active compound. A suitable carrier is, for example, stable, e.g., incapable of reacting with other ingredients in the carrier. In one example, the carrier does not produce significant local or systemic adverse effect in recipients at the dosages and concentrations employed for treatment.

Suitable carriers for the present disclosure include those conventionally used, e.g., water, saline, aqueous dextrose, lactose, Ringer's solution, a buffered solution, hyaluronan and glycols are exemplary liquid carriers, particularly (when isotonic) for solutions. Suitable pharmaceutical carriers and excipients include starch, cellulose, glucose, lactose, sucrose, gelatin, malt, rice, flour, chalk, silica gel, magnesium stearate, sodium stearate, glycerol monostearate, sodium chloride, glycerol, propylene glycol, water, ethanol, and the like.

In another example, a carrier is a media composition, e.g., in which a cell is grown or suspended. Such a media composition does not induce any adverse effects in a subject to whom it is administered. Exemplary carriers and excipients do not adversely affect the viability of a cell and/or the ability of a cell to treat or prevent disease.

In one example, the carrier or excipient provides a buffering activity to maintain the cells and/or soluble factors at a suitable pH to thereby exert a biological activity, e.g., the carrier or excipient is phosphate buffered saline (PBS). PBS represents an attractive carrier or excipient because it interacts with cells and factors minimally and permits rapid release of the cells and factors, in such a case, the composition of the disclosure may be produced as a liquid for direct application to the blood stream or into a tissue or a region surrounding or adjacent to a tissue, e.g., by injection.

Compositions of the disclosure may be cryopreserved. Cryopreservation of mesenchymal lineage precursor or stem cells can be carried out using slow-rate cooling methods or ‘fast’ freezing protocols known in the art. Preferably, the method of cryopreservation maintains similar phenotypes, cell surface markers and growth rates of cryopreserved cells in comparison with unfrozen cells.

The cryopreserved composition may comprise a cryopreservation solution. The pH of the cryopreservation solution is typically 6.5 to 8, preferably 7.4.

The cyropreservation solution may comprise a sterile, non-pyrogenic isotonic solution such as, for example, PlasmaLyte ATM. 100 mL of PlasmaLyte ATM contains 526 mg of sodium chloride, USP (NaCl); 502 mg of sodium gluconate (C6H11NaO7); 368 mg of sodium acetate trihydrate, USP (C2H3NaO2·3H2O); 37 mg of potassium chloride, USP (KCl); and 30 mg of magnesium chloride, USP (MgCl2·6H2O). It contains no antimicrobial agents. The pH is adjusted with sodium hydroxide. The pH is 7.4 (6.5 to 8.0).

The cryopreservation solution may comprise Profreeze™. The cryopreservation solution may additionally or alternatively comprise culture medium, for example, αMEM.

To facilitate freezing, a cryoprotectant such as, for example, dimethylsulfoxide (DMSO), is usually added to the cryopreservation solution. Ideally, the cryoprotectant should be nontoxic for cells and patients, nonantigenic, chemically inert, provide high survival rate after thawing and allow transplantation without washing. However, the most commonly used cryoprotector, DMSO, shows some cytotoxicity. Hydroxylethyl starch (HES) may be used as a substitute or in combination with DMSO to reduce cytotoxicity of the cryopreservation solution.

The cryopreservation solution may comprise one or more of DMSO, hydroxyethyl starch, human serum components and other protein bulking agents. In one example, the cryopreserved solution comprises Plasma-Lyte A (70%), DMSO (10%), HSA (25%) solution, the HSA solution comprising 5% HSA and 15% buffer.

In an example, the cryopreservation solution may further comprise one or more of methycellulose, polyvinyl pyrrolidone (PVP) and trehalose.

The cryopreserved composition may be thawed and administered directly to the subject or added to another solution, for example, comprising hyaluronic acid. Alternatively, the cryopreserved composition may be thawed and the mesenchymal lineage precursor or stem cells resuspended in an alternate carrier prior to administration.

The compositions described herein may be administered alone or as admixtures with other cells. The cells of different types may be admixed with a composition of the disclosure immediately or shortly prior to administration, or they may be co-cultured together for a period of time prior to administration.

In one example, the composition comprises an effective amount or a therapeutically or prophylactically effective amount of mesenchymal lineage precursor or stem cells and/or progeny thereof and/or soluble factor derived therefrom. For example, the composition comprises about 1×10⁵ stem cells to about 1×10⁹ stem cells or about 1.25×10³ stem cells to about 1.25×10⁷ stem cells/kg (80 kg subject). The exact amount of cells to be administered is dependent upon a variety of factors, including the age, weight, and sex of the subject, and the extent and severity of the disorder being treated.

Despite the number of cells provided in the composition, in an example, 50×10⁶ to 200×10⁷ cells are administered. In other examples, 60×10⁶ to 200×10⁶ cells or 75×10⁶ to 150×10⁶ cells are administered. In an example, 75×10⁶ cells are administered. In another example, 150×10⁶ cells are administered.

In an example, the composition comprises greater than 5.00×10⁶ viable cells/mL. In another example, the composition comprises greater than 5.50×10⁶ viable cells/mL. In another example, the composition comprises greater than 6.00×10⁶ viable cells/mL. In another example, the composition comprises greater than 6.50×10⁶ viable cells/mL. In another example, the composition comprises greater than 6.68×10⁶ viable cells/mL.

In an example, the mesenchymal lineage precursor or stem cells comprise at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 99% of the cell population of the composition.

In an example, the composition may optionally be packaged in a suitable container with written instructions for a desired purpose.

In an example, MLPSCs may be administered to a wall of a subjects gastrointestinal tract. In an example, MLPSCs can be administered to a site of inflammation in a subjects gastrointestinal tract wall. For example, MLPSCs can be administered into a site of inflammation in a subjects gastrointestinal tract wall. In these examples, the site of inflammation may be endoscopicaly confirmed prior to administration. For example, endoscopic confirmation can be based on visual inspection by a trained physician and/or histological analysis of endoscopic biopsy. In another example, compositions of the disclosure are administered intravenously.

In an example, the compositions described herein may be administered as a single dose.

In some examples, the compositions described herein may be administered over multiple doses. For example, at least 2, at least 3, at least 4, at least 5, at least 6, at least 7, at least 8, at least 9, at least 10 doses.

In an example, MLPSCs may be administered intravenously. In another example, MLPSCs are administered once weekly. For example, MLPSCs can be administered once weekly every two weeks. In an example, MLPSCs can be administered once monthly. In an example, two doses of MLPSCs are administered once weekly. In another example, two doses of MLPSCs are administered once weekly every two weeks. For example, two doses of MLPSCs can be administered once weekly every two weeks before subsequent doses are administered once monthly. In an embodiment of this example, doses are administered monthly for a further one, two, three, four, five, six, seven or more months.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

All publications discussed and/or referenced herein are incorporated herein in their entirety.

The present application claims priority from AU2021904222 filed 23 Dec. 2021, the disclosures of which are incorporated herein by reference.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed before the priority date of each claim of this application.

### EXAMPLES

### Composition

The composition consists of human bone marrow-derived allogeneic mesenchymal stem cells (MSCs) isolated from bone mononuclear cells, expanded ex vivo, and cryopreserved.

### MAGIC Biomarker Algorithm Probability (MAP)

The MAGIC Algorithm Probability (MAP) combines the serum concentrations of two biomarkers, Reg3α and ST2, into a single value that predicts long-term outcomes such as response to therapy after 28 days and 6-month non-relapse mortality (NRM). MAP is calculated by:

$$log 10 [ - log 10 ( 1 - \hat{p} ) ] = - 11.263 + 1.844 ( log 10 ST 2 ) + 0.577 ( log 10 REG 3 α )$$

where \hat{p} is the predicted probability of 6-month NRM (Hartwell et al.; Major-Monfried et al.). A MAP score of ≥0.291 is a validated threshold that identifies GvHD patients with the most severe disease who are at high risk of non-response to treatment and death.

MAP estimates the damage to GI crypts, a key biologic metric of GI tract health. Patients with high MAPs (i.e. ≥0.291) have significant GI crypt damage and a very low probability of achieving a clinical response to additional therapy within 28 days. These patients have a low overall survival rate at 6 months on the order of 20%. Patients who have steroid-refractory GvHD and low MAPs have less extensive GI crypt damage and are very likely to be slow responders, and may in fact not need additional treatment. The overall survival after 6 months of treatment for these patients with low MAPs is approximately 80%. Accordingly, transitioning patients from high MAP (i.e. ≥0.291) to low MAP (i.e. <0.291) is likely of clinical importance, at the very least in terms of improved survival outcomes.

### Patients

25 cell therapy treated children with steroid-refractory acute GvHD from a 54-patient paediatric study GvHD001/02 with paired baseline and Day 28 serum samples were compared to 27 closely matched paediatric patients with steroid-refractory acute GvHD who participated in a prospective GvHD natural history study. All 52 evaluated patients in both groups had GvHD that had either worsened within 3 days, not responded within 7 days, or progressed after an initial response to treatment with systemic steroids and was Grade B-D GvHD according to the IBMTR severity scale at the time second line treatment was initiated. Control patients were included if they met the study criteria for GvHD severity at the time they received second line therapy and had serum samples available when second line therapy was initiated.

Patients with steroid-refractory grade B skin-only disease (i.e., stage 2 skin rash only) were excluded from the study. All biomarker assays were conducted by investigators blinded to clinical outcomes.

Table 4 shows a comparison of baseline characteristics at the time of second treatment. There were no significant differences between the two groups for age, time to second-line treatment, Minnesota high-risk or multi-organ involvement. There was a significantly higher percentage of clinically severe (grade III/V) acute GvHD in the cell therapy group 20/25 (80%) v 16/27 (59%), p=0.02). Therefore, a very large percentage of these patients had severe, high-risk clinical disease at the initiation of cell therapy.

Second-line treatments in the control group were determined by the treating physician and included extracorporeal photopheresis, etanercept, infliximab, ruxolitinib, anti-thymocyte globulin, mycophenolate, alemtuzumab, basiliximab, and tocilizumab.

TABLE 4
Baseline patient characteristics

| | MAGIC controls | Remestemcel-L | p- |
|---|---|---|---|
| Characteristic | (n = 27) | (n = 25) | value |
| Median age, years (range) | 6 (0-17) | 10 (0-16) | 0.36 |
| Median days between initial | 10 (4-28) | 10 (4-26) | 0.53 |
| treatment and 2^{nd} line (range) | | | |
| Grade at 2^{nd} line, % (n) | | | 0.02 |
| II | 41% (11) | 20% (6) | |
| III/IV | 59% (16) | 80% (20) | |
| Minnesota high risk | 56% (15) | 76% (19) | 0.12 |
| Multi organ involvement | 44% (12) | 36% (9) | 0.39 |

### Results

Serum concentrations of Reg3α and ST2 were determined by ELISA and MAP was calculated for all 52 patients. At baseline the mean MAPs were similar between the study patients treated with cell therapy vs MAGIC controls (0.283±0.17 vs 0.262±0.20, p=0.67). 48% (12/25) cell therapy patients and 37% (10/27) of MAGIC control patients were high risk by baseline, MAP ≥0.291.

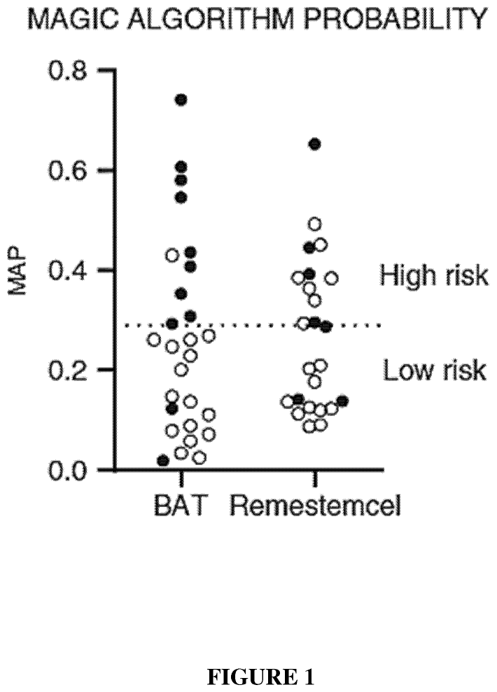

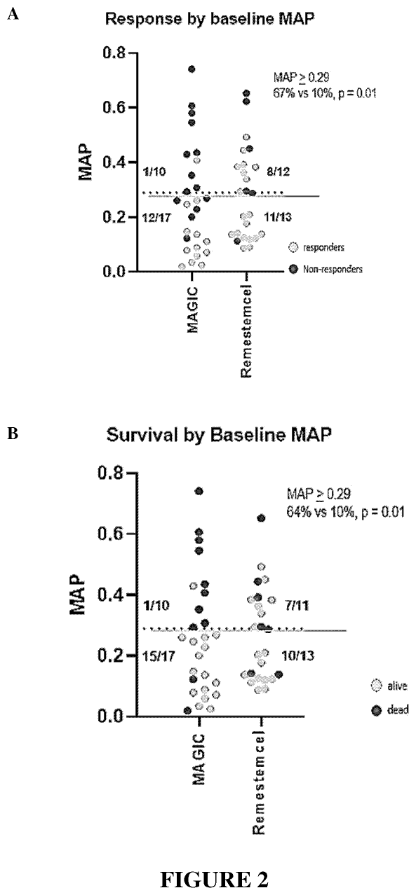

The clinical response after 28 days in the cell therapy group (18/25, 72%) was higher than in the control group (13/27, 48%). Surprisingly, survival at day +180 was significantly better for patients with a high-risk MAP treated with cell therapy compared to high-risk control patients who received best available therapy (7/11, 64% vs 1/10, 10%, p=0.01; FIG. 1). Consistent with these survival results, 8/12 study patients treated with cell therapy, who were high risk by biomarkers, achieved a clinical response by day 28 of cell therapy, compared to only 1/10 MAGIC patients who received the best available treatment (67% vs 10%, p=0.01) (FIG. 2). These data suggest that cell therapy is particularly effective at treating GvHD in patients with the most severe disease. This is an important finding as there is a significant unmet clinical need in these patients, in particular in view of them being refractory to one or more therapies (e.g. steroids) and at high risk of mortality.

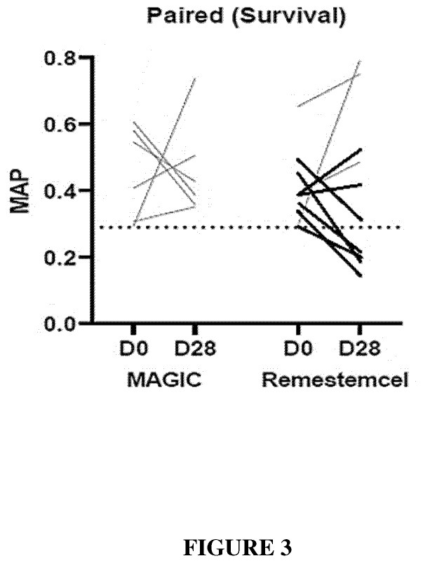

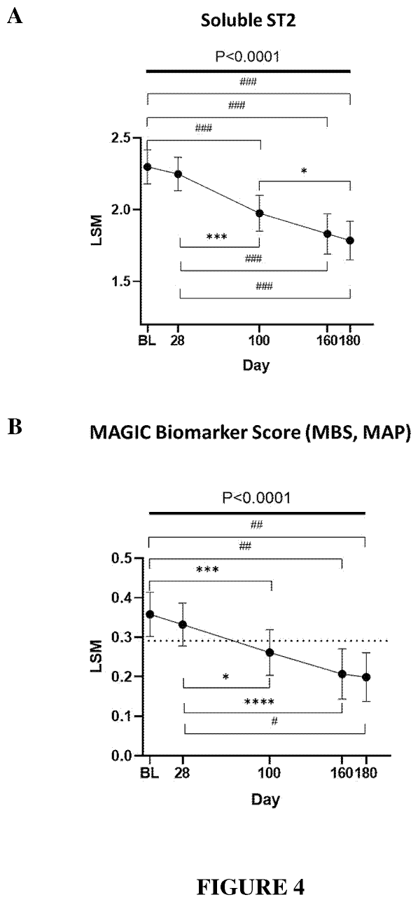

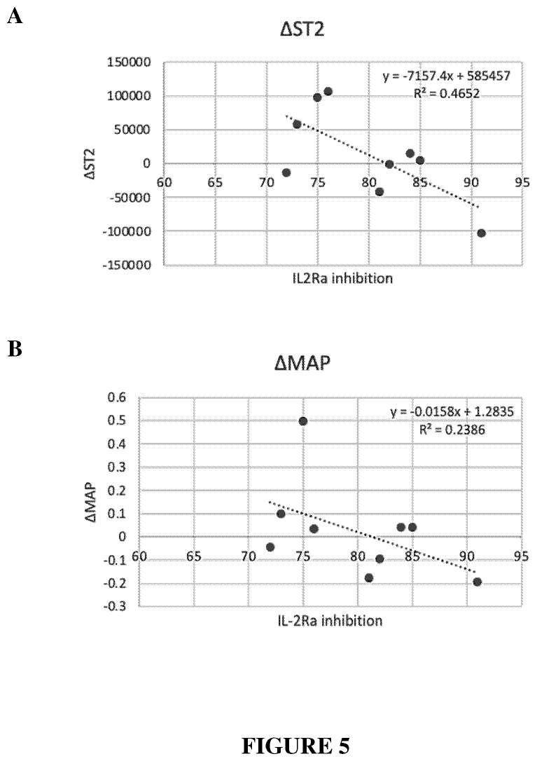

In addition, improved survival in cell therapy treated children with baseline MAP ≥0.29 is associated with decreased MAP levels over 28 days (FIG. 3). Without wishing to bound be a particular theory, the decrease in MAP levels among those with baseline MAP ≥0.291 suggests that cell therapy may be acting to heal crypt damage in severely ill patients, potentially, at least in part, by promoting an anti-inflammatory response. As shown in FIG. 4, cell therapy was also associated with a durable reduction in both ST2 biomarker levels and MAP. By day 100, patients MAP score had decreased below 0.291, continually decreasing through day 180. These data indicate that treatment with cell therapy unexpectedly improves disease severity as determined by MAP and that this treatment response is durable. Interestingly, the level of IL2R inhibition by stem cells correlated with reduction over 28 days in circulating ST2 levels and MAP scores in GvHD patients (FIG. 5).

The above referenced results from cell therapy are striking when viewing against outcomes for control patients with high MAP. For control patients from the MAGIC database who received best available therapy, 15 of 17 (88%) patients with low MAPs survived 6 months, whereas only 1 of 10 (10%) with high MAPs survived. In contrast, survival in patients with a high MAP who received cell therapy (7/11, 64%) was high and significantly better compared to the controls with high MAPs. Survival in patients with a low MAP who received cell therapy (11/13, 85%). As noted above, this pattern was also seen in the response rate of patients 28 days after therapy. The fact that nearly two thirds of study patients who were categorized as high risk by biomarkers responded to cell therapy and survived 6 months is surprising and clinically important. These data underpin a rationale for administering cell therapy to patients with severe GvHD, in particular in view of the improved survival associated with the same.

Stratifying patients on the basis of having received stem cell product with in vitro attribute of mean % IL-2Rα inhibition >median (median=81% inhibition) or ≤median showed a positive association between % IL-2Rα inhibition and Day 180 survival (85% vs. 54%, p=0.01). This was preceded by a greater duration of the Day 28 OR among responders who received product with higher IL-2Rα inhibition. The relationship between greater survival and mean % IL-2Rα inhibition above the median vs below the median was most evident in patients with the most severe form of the disease, and at highest risk for death: those classified as Minnesota high risk (Day 180 OS 89% vs 50%, p=0.01), MAGIC algorithm probability (MAP) score ≥0.29 (Day 180 OS 100% vs. 17%, p=0.003) or Grade D disease (Day 180 OS 91% vs. 50%, p=0.03).

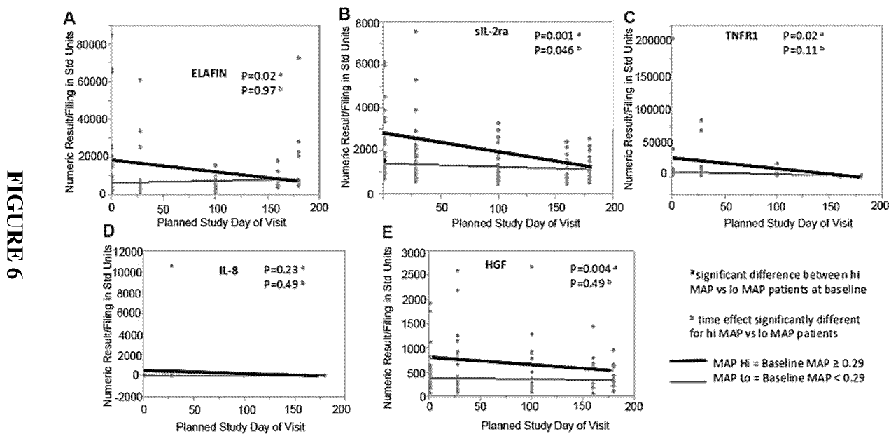

Product lot potency (inhibitory activity on IL-2Rα expression on activated PBMC in vitro), correlated with progressively decreasing inflammatory biomarkers in vivo. This was most evident in those children with highest baseline levels of inflammatory biomarkers, defined prospectively on the basis of having high MAP scores (FIG. 6). There was a significant relationship between % IL-2Rα inhibition in vitro as a measure of a lot's potency and reduction in CD3+CD4+CD25+HLA−DR+activated T cell phenotype, from screening to Day 28 (p=0.009).

The inhibitory activity of stem cell product on IL-2Rα levels in cultures of activated PBMC correlates with in vivo bioactivity in pediatric aGVHD as indicated by decreases in inflammatory biomarkers, reductions in circulating levels of inflammatory biomarkers and activated T cells and improved survival.

### REFERENCES

Hartwell et al., (2017) JCI Insight.; 2(3):e89798.
Glucksberg et al., (1974); Transplantation; 18, 295-304.
MacMillan et al., (2015) Biol Blood Marrow Transplant; 21(4): 761-767.
Major-Monfried et al., (2018) Blood; 131(25):2846-2855
Rowlings et al., (1997) British Journal of Haematology; 97, 855-864
Thomas et al., (1975) New England Journal of Medicine; 292, 895-902.

1. A method for treating Graft versus Host Disease (GvHD) in a subject, the method comprising administering to the subject a composition comprising mesenchymal lineage precursor or stem cells (MLPSC), wherein the subject has severe GvHD as determined by MAGIC Algorithm Probability (MAP).

2. A method of selecting Graft versus Host Disease (GvHD) patients for treatment with mesenchymal lineage precursor or stem cells (MLPSC), the method comprising i) determining or having determined a subject's MAGIC Algorithm Probability (MAP) ii) selecting a subject having MAP indicative of severe GvHD for treatment, preferably, wherein the treatment comprises administering a composition comprising MLPSCs.

3. A method of reducing the risk of mortality in a subject with Graft versus Host Disease (GvHD), wherein the subject has severe GvHD as measured by MAGIC Algorithm Probability (MAP), the method comprising administering to the subject a composition comprising mesenchymal lineage precursor or stem cells (MLPSC), preferably wherein mortality is 6-month non-relapse mortality (NRM).

4. The method according to any one of claims 1 to 3, wherein the subject's MAP is greater than 0.16, preferably greater than 0.2, more preferably greater than 0.29, alternatively between 0.16 and 0.35.

5. The method according to any one of claims 1 to 3, wherein the subject's MAP is greater than or equal to (≥) 0.29.

6. The method according to any one of claims 1 to 5, wherein the subject is steroid refractory.

7. The method according to any one of claims 1 to 6, wherein the subject has acute Graft versus Host Disease (aGvHD).

8. The method according to any one of claims 1 to 7, wherein the subject is a paediatric subject.

9. The method according to any one of claims 1 to 8, wherein the subject has very low probability of achieving a clinical response to primary therapy within 28 days and/or has a high risk of 6 month non-relapse mortality.

10. The method according to any one of claims 1 to 9, wherein the subject has a 6 month non-relapse mortality risk of ≥70%, ≥80%, or ≥90%.

11. The method according to any one of claims 1 to 10, wherein the subject's GvHD has worsened within 3 days of primary therapy or not responded within 7 days of a primary therapy.

12. The method according to claim 11, wherein the primary therapy is systemic steroids.

13. The method according to any one of claims 1 to 12, wherein the subject is classified as one or more of the following:
Grade B, C, or D according to the IBMTR severity scale;
Grade II GvHD according to the Glucksberg severity scale;
Grade IIII/V GvHD according to the Glucksberg severity scale;
Minnesota high risk GvHD.

14. The method according to any one of claims 1 to 13, wherein the subject has multi-organ involvement.

15. The method according to any one of claims 1 to 14, wherein the subject has at least a partial response after 28 days of treatment.

16. The method according to any one of claims 1 to 15, wherein the subject has at least a partial response at least 28 to 180 days after treatment.

17. The method of claim 15 or 16 wherein a partial response is characterized by one or more or all of:
Reduction in Skin % BSA score of at least one point;
Reduction in mouth score of at least one point;
Reduction in eye score of at least one point;
Reduction in skin features score of at least one point;
Reduction in gastrointestinal tract score of at least one point;
Reduction in liver score of at least one point;
Reduction in lung symptom score of at least one point;
Reduction in lung FEV1 score of at least one point;
Reduction in joints and fascia score of at least one point;
Reduction in genital tract score of at least one point.

18. The method of claim 15 or 16 wherein a partial response is characterized by one or more or all of:
Reduction in Skin % BSA score of at least one point;
Reduction in mouth score of at least one point;
Reduction in eye score of at least one point.

19. The method according to any one of claims 1 to 18, wherein treatment increases the probability of the subject's survival.

20. The method according to any one of claims 1 to 19, wherein treatment increases the probability of the subject surviving for at least 100 days after initiation of treatment, preferably at least 180 days.

21. The method according to any one of claims 1 to 20, wherein treatment decreases the subject's risk of 6 month non-relapse mortality to between 40% and 80%, preferably at least 60%.

22. The method according to claim 21, wherein the subject's risk of 6 month non-relapse mortality is decreased relative to a subject who does not receive MLPSCs.

23. The method of claim 22, wherein the subject that does not receive MLPSCs receives best available therapy, preferably, wherein best available therapy is selected from one of more of the group consisting of extracorporeal photopheresis, etanercept, infliximab, ruxolitinib, anti-thymocyte globulin, mycophenolate, alemtuzumab, basiliximab, and tocilizumab.

24. The method according to any one of claims 1 to 23, wherein treatment reduces the subject's ST2 levels, preferably wherein treatment also reduces the subject's level of one or more inflammatory biomarkers selected from the group consisting of ELAFIN, sIL-2ra, TNFR1, IL-8 and HGF.

25. The method according to any one of claims 1 to 24, wherein treatment decreases the subject's MAP, preferably wherein the subject's MAP is decreased to <0.29.

26. The method according to claim 25, wherein treatment decreases the subject's MAP by day 28.

27. The method according to claim 25, wherein treatment decreases the subject's MAP to <0.29 by day 100.

28. The method according to any one of claims 25 to 27, wherein the decrease in the subject's MAP is sustained at day 100, at day 160, at day 180.

29. The method according to any one of claims 1 to 28 which comprises the steps of:
i) determining or having determined the subject's Reg3α and ST2 levels;
ii) calculating or having calculated the subject's MAP according to the subject's Reg3α and ST2 levels;
iii) selecting a subject for treatment with MLPSCs, wherein the subject's MAP is ≥0.29; and
iv) administering to the subject a composition comprising MLPSCs.

30. The method according to any one of claims 1 to 29, wherein the MLPSCs are STRO-1+.

31. The method according to any one of claims 1 to 30, wherein the MLPSCs are allogeneic.

32. The method according to any one of claims 1 to 31, wherein the cells are culture expanded.

33. The method according to claim 32, wherein the cells are TNAP+before they are culture expanded.

34. The method according to any one of claims 1 to 33, wherein the cells have been cryopreserved.

35. The method according to any one of claims 1 to 34 which comprises administering between 1×10⁷ and 2×10⁸ cells.

36. The method according to any one of claims 1 to 35, wherein the composition further comprises Plasma-Lyte A, dimethyl sulfoxide (DMSO), human serum albumin (HSA).

37. The method according to any one of claims 1 to 36, wherein the composition further comprises Plasma-Lyte A (70%), DMSO (10%), HSA (25%) solution, the HSA solution comprising 5% HSA and 15% buffer.

38. The method according to any one of claims 1 to 37, wherein the composition comprises greater than 6.68×10⁶ viable cells/mL.

39. The method according to any one of claims 1 to 38, wherein the composition comprises human bone marrow-derived allogeneic mesenchymal precursor cells (MPCs) isolated from bone mononuclear cells with anti-STRO-3 antibodies, expanded ex vivo, and cryopreserved.

40. The method according to claim 1 to 37, wherein the MLPSCs are mesenchymal stem cells (MSCs).

41. The method according to any one of claims 1 to 40, wherein the subject has previously received another therapy.

42. The method according to claim 41, wherein the therapy is selected from the group consisting of steroids, extracorporeal photopheresis, etanercept, infliximab, ruxolitinib, anti-thymocyte globulin, mycophenolate, alemtuzumab, basiliximab, and tocilizumab.

43. The method according to any one of claims 1 to 42, wherein the composition is administered to the gastrointestinal tract wall of the subject.

44. The method according to claim 43, wherein the composition is administered to a site of inflammation in the subject's gastrointestinal tract wall.

45. The method according to any one of claims 1 to 42, wherein the composition is administered intravenously.

46. The method according to any one of claims 43 to 45, wherein the subject receives at least two doses.

47. The method according to claim 46, wherein the subject receives at least 2, 3, 4, 5, 6, 7, 8, 9 or 10 doses.

48. The method according to claim 46 or 47, wherein the first two doses are administered weekly for two weeks.

49. The method according to claim 46 or 46, wherein the first two doses are administered weekly every two weeks.

50. The method of claim 49, wherein third and subsequent doses are administered monthly.
